(19) United States
(12) Patent Application Publication
(10) Pub. No.: US 2026/0009087 A1
(43) Pub. Date: Jan. 8, 2026

(54) COMBINATION OF BLOCKERS, KIT AND METHOD FOR DETECTING DRUG RESISTANCE OF MYCOBACTERIUM TUBERCULOSIS

(71) Applicant: Delta Electronics (Shanghai) Co., Ltd., Shanghai (CN)

(72) Inventors: Xiao-Zhi ZHAO, Shanghai (CN); Quan-Ke LONG, Shanghai (CN); Jiao-Jiao LI, Shanghai (CN); Wei-Shi ZHANG, Shanghai (CN)

(21) Appl. No.: 19/257,483

(22) Filed: Jul. 2, 2025

(30) Foreign Application Priority Data
Jul. 2, 2024 (CN) 202410884005.9

(51) Int. Cl. C12Q 1/689; C12Q 1/6827; C12Q 1/686

(52) U.S. Cl. CPC C12Q 1/689; C12Q 1/6827; C12Q 1/686; C12Q 2600/156

(57) ABSTRACT

A combination of blockers for detecting drug resistance of Mycobacterium tuberculosis, including a first peptide nucleic acid (PNA) blocker, a second PNA blocker, a third PNA blocker, and a fourth PNA blocker. The first PNA blocker covers codons 511 and 513 of rpoB gene. The second PNA blocker covers codon 516 of rpoB gene. The third PNA blocker covers codon 526 of rpoB gene. The fourth PNA blocker covers codons 531 and 533 of rpoB gene. The sequences of the first, second, third, and fourth PNA blockers respectively match the wild-type sequences of the corresponding regions of rpoB gene. A kit and method for detecting drug resistance of Mycobacterium tuberculosis are also provided.

### CROSS-REFERENCE TO RELATED APPLICATION

This application claims priority to China Application Serial Number 202410884005.9, filed Jul. 2, 2024, which is herein incorporated by reference in its entirety.

The Sequence Listing associated with this application is filed in electronic format via EFS-Web and is hereby incorporated by reference into the specification in its entirety. The name of the text file containing the Sequence Listing is NP-36023-US_SEQ_LIST.txt. The size of the text file is 22,992 B, and the text file was created on Jun. 27, 2025.

### BACKGROUND

### Field of Invention

The present disclosure relates to a combination of blockers, a kit, and a method for detecting drug resistance of Mycobacterium tuberculosis using quantitative PCR.

### Description of Related Art

Tuberculosis (TB) is the second most deadly infectious disease after COVID-19, ranking as the 13th leading cause of death worldwide. It is also the leading cause of death among people infected with HIV and a major cause of death related to antimicrobial resistance. On Oct. 27, 2022, the World Health Organization (WHO) released the latest “Global Tuberculosis Report 2022,” which showed that there were 10.6 million new TB cases globally in 2021, with an incidence rate of 134 per 100,000 people. Currently, drugs used for TB treatment include rifampicin, isoniazid, ethambutol, pyrazinamide, streptomycin, etc., but drug resistance is severe. According to the report, the burden of drug-resistant TB (DR-TB) increased during 2020-2021. In 2021, there were 450,000 new cases of rifampicin-resistant TB (RR-TB). The types of drugs available for TB treatment are limited, and there is cross-resistance between first-line and second-line TB drugs. Additionally, almost no new treatment drugs introduced in recent years, leading to poor efficacy and unfavorable prognosis. TB brings immense mental and financial pressure to patients and their families, while also causing a significant economic burden to the country.

In clinical practice, the traditional method for drug susceptibility testing of drug-resistant tuberculosis is still based on culture-based drug sensitivity testing. This method can simultaneously detect the resistance levels to first-line and second-line drugs, but the results may be easily affected by factors such as drug concentration, the inoculum size of Mycobacterium tuberculosis (MTB) and the viability of TB, leading to inaccurate results. Moreover, the culture-based methods are time-consuming, taking about 1 month on average. On the basis of traditional culture methods, a rapid liquid culture system for Mycobacteria has been developed. This system shortens the conventional culture time from an average of 1 month to 8 to 10 days. However, this system requires specialized equipment, and the imported products for this system are expensive. In addition, the minimum inhibitory concentration (MIC) testing method for Mycobacterium tuberculosis can shorten the testing time to 1 to 2 weeks compared to traditional culture methods. However, in practical use of this method, the MIC of some drugs may be difficult to interpret. In contrast, molecular diagnostic-based methods for detecting drug resistance can shorten the detection time to within one day, greatly improving detection speed and efficiency.

Therefore, the Chinese “Expert Consensus on Detection of Mycobacterium tuberculosis Drug Resistance” recommends: (1) Combining phenotypic drug susceptibility testing (DST) with molecular DST for complementary advantages. (2) Using molecular DST methods to directly detect clinical specimens from TB patients to rapidly screen for MTB and rifampicin-resistant MTB. (3) Using molecular DST methods to directly detect smear-positive clinical samples. (4) Handling of specimens from smear-negative retreatment TB patients: it is recommended to first conduct molecular testing; if the nucleic acid test is positive, further molecular drug susceptibility testing (DST) can provide clear results on drug-resistant genotypes for some smear-negative specimens at an early stage.

WHO also recommends rapid molecular diagnostic testing to achieve earlier and more accurate diagnosis of TB and drug-resistant TB, and considers the accessibility of such diagnostic testing a crucial aspect of strengthening TB laboratory efforts in the “End TB” Strategy. However, the use of such rapid testing technology remains very limited. Of the 6.4 million new TB cases diagnosed in 2021, only 38% used WHO-recommended rapid molecular testing, slightly higher than 33% in 2020 and 28% in 2019.

Currently, the main players in the market for molecular diagnostic methods for DR-TB detection include Cepheid, Zhishan Biological Technology, Yaneng Bioscience, Daan Gene, and InnowaveDx. Among them, Cepheid occupies the mainstream market with its integrated and portable characteristics of molecular point-of-care testing (POCT). Cepheid's products are designed for detecting rifampicin-resistant TB. Zhishan Biological Technology focuses on detecting drug resistance genes for other clinical TB treatments, and the drugs include isoniazid, ethambutol, quinolones, etc. With this advantage, Zhishan Biological Technology has become the second-largest domestic TB drug resistance testing brand after Cepheid. Yaneng Bioscience and Daan Gene provide testing solutions to the market by combining a Mycobacterium tuberculosis DNA detection kit with a rifampicin resistance gene detection kit. InnowaveDx's drug resistance detection product is a recently approved kit for detecting Mycobacterium tuberculosis and rifampicin resistance.

The current molecular testing methods for drug-resistant TB mentioned above are complex in operation, require expensive equipment, and have high costs, which pose many challenges for practical clinical use. Additionally, the detection sensitivity and specificity may be insufficient. In addition, the mutation sites included in the testing are not comprehensive, which may lead to missed detections in clinical practice. Furthermore, there are currently not many detection methods based on quantitative PCR technology that directly target mutated genes. One key issue that needs to be addressed is the impact of non-specific amplification caused by wild-type (non-mutated) templates on the detection results, which may lead to potential false positive results.

### SUMMARY

Embodiments of the present disclosure address the issue of non-specific amplification arising from wild-type (non-mutated) templates during drug resistance testing of MTB using quantitative PCR technology, thereby reducing false positive results. In some embodiments, a combination of peptide nucleic acid (PNA) blockers is provided to comprehensively cover the mutation sites in the drug resistance-determining region of rpoB and match the wild-type sequences of these mutation sites. In some embodiments, a quantitative PCR detection kit and detection method based on the combination of the blockers are also included.

Some embodiments of the present disclosure provide a combination of blockers for detecting drug resistance of Mycobacterium tuberculosis, including a first PNA blocker, a second PNA blocker, a third PNA blocker, and a fourth PNA blocker. The first PNA blocker covers codons 511 and 513 of rpoB gene. The second PNA blocker covers codon 516 of rpoB gene. The third PNA blocker covers codon 526 of rpoB gene. The fourth PNA blocker covers codons 531 and 533 of rpoB gene. The sequences of the first, second, third, and fourth PNA blockers respectively match the wild-type sequences of the corresponding regions of rpoB gene.

In some embodiments, the first PNA blocker includes the sequence of SEQ ID NO: 1 or a derivative thereof, the second PNA blocker includes the sequence of SEQ ID NO: 2 or a derivative thereof, the third PNA blocker includes the sequence of SEQ ID NO: 3 or a derivative thereof, and the fourth PNA blocker includes the sequence of SEQ ID NO: 4 or a derivative thereof.

In some embodiments, each of the first, second, third, and fourth PNA blockers has 14 to 18 bases.

In some embodiments, the sequence of the first PNA blocker is SEQ ID NO: 1, the sequence of the second PNA blocker is SEQ ID NO: 2, the sequence of the third PNA blocker is SEQ ID NO: 3, and the sequence of the fourth PNA blocker is SEQ ID NO: 4.

In some embodiments, the first, second, third, and fourth PNA blockers do not contain fluorescent modification groups.

Some embodiments of the present disclosure provide a kit for detecting drug resistance of Mycobacterium tuberculosis, including at least one set of primer pair, a combination of blockers, and at least one mutation detection probe. The at least one set of primer pair is used to amplify a segment of rpoB gene. The combination of blockers includes a plurality of PNA blockers covering codons 511, 513, 516, 526, 531, and 533 in the drug resistance-determining region of rpoB gene, and the sequences of these PNA blockers respectively match the wild-type sequences of the corresponding regions. The at least one mutation detection probe has a single-base mismatch and is used to detect mutations in at least one of codons 511, 513, 516, 526, 531, and 533 in the drug resistance-determining region of rpoB gene.

In some embodiments, the at least one set of primer pair may be one set of primer pair, two sets of primer pairs (e.g., for nested PCR), or more sets of primer pairs.

In some embodiments, the kit for detecting drug resistance of Mycobacterium tuberculosis further includes an internal control primer pair and an internal control detection probe.

In some embodiments, in the kit for detecting drug resistance of Mycobacterium tuberculosis, the PNA blockers includes: a first PNA blocker, a second PNA blocker, a third PNA blocker, and a fourth PNA blocker. The first PNA blocker covers codons 511 and 513 of rpoB gene. The second PNA blocker covers codon 516 of rpoB gene. The third PNA blocker covers codon 526 of rpoB gene. The fourth PNA blocker covers codons 531 and 533 of rpoB gene.

In some embodiments, in the kit for detecting drug resistance of Mycobacterium tuberculosis, the first PNA blocker includes the sequence of SEQ ID NO: 1 or a derivative thereof, the second PNA blocker includes the sequence of SEQ ID NO: 2 or a derivative thereof, the third PNA blocker includes the sequence of SEQ ID NO: 3 or a derivative thereof, and the fourth PNA blocker includes the sequence of SEQ ID NO: 4 or a derivative thereof.

In some embodiments, in the kit for detecting drug resistance of Mycobacterium tuberculosis, the sequence of the first PNA blocker is SEQ ID NO: 1, the sequence of the second PNA blocker is SEQ ID NO: 2, the sequence of the third PNA blocker is SEQ ID NO: 3, and the sequence of the fourth PNA blocker is SEQ ID NO: 4.

In some embodiments, in the kit for detecting drug resistance of Mycobacterium tuberculosis, each of the at least one detection probe is used to detect a mutation site in rpoB gene, and the mutation site is one of L511P, Q513K, Q513P, D516V, D516Y, H526D, H526L, H526R, H526Y, S531L, S531W, and L533P.

In some embodiments, the PNA blockers do not contain fluorescent groups, and the 5′ and 3′ ends of the at least one detection probe are modified with a fluorescent reporter group and a quencher group, respectively.

Some embodiments of the present disclosure provide a method for detecting drug resistance of Mycobacterium tuberculosis, including: adding template DNA, at least one set of primer pair, a combination of blockers, and at least one mutation detection probe to a reaction system to perform a PCR reaction, wherein the combination of blockers includes a plurality of PNA blockers covering codons 511, 513, 516, 526, 531, and 533 in the drug resistance-determining region of rpoB gene, and the sequences of these PNA blockers match the wild-type sequences of the corresponding regions. The at least one mutation detection probe is used to detect a mutation site in at least one of codons 511, 513, 516, 526, 531, and 533 in the drug resistance-determining region of rpoB gene. The presence of a rifampicin resistance related mutation in the template DNA is determined based on the reaction signals from the PCR reaction.

In some embodiments, the PCR reaction is a nested PCR, including: a first round of PCR reaction and a second round of PCR reaction. The first round of PCR reaction is conducted for 8 to 15 cycles. The second round of PCR reaction is conducted for 35 to 50 cycles, wherein the annealing/extension stage of the second round of PCR is divided into a first step and a second step, and the temperature of the first step is higher than the temperature of the second step.

In some embodiments, in the method for detecting drug resistance of Mycobacterium tuberculosis, the plurality of PNA blockers include: a first PNA blocker, a second PNA blocker, a third PNA blocker, and a fourth PNA blocker. The first PNA blocker covers codons 511 and 513 of rpoB gene. The second PNA blocker covers codon 516 of rpoB gene. The third PNA blocker covers codon 526 of rpoB gene. The fourth PNA blocker covers codons 531 and 533 of rpoB gene. The sequences of the first, second, third, and fourth PNA blockers respectively match the wild-type sequences of the corresponding regions of rpoB gene.

In some embodiments, the first PNA blocker includes the sequence of SEQ ID NO: 1 or a derivative thereof, the second PNA blocker includes the sequence of SEQ ID NO: 2 or a derivative thereof, the third PNA blocker includes the sequence of SEQ ID NO: 3 or a derivative thereof, and the fourth PNA blocker includes the sequence of SEQ ID NO: 4 or a derivative thereof.

In some embodiments, in the method for detecting drug resistance of Mycobacterium tuberculosis, each of the at least one mutation detection probe is used to detect a mutation site in rpoB gene, and the mutation site is one of L511P, Q513K, Q513P, D516V, D516Y, H526D, H526L, H526R, H526Y, S531L, S531W, and L533P.

In some embodiments, the at least one mutation detection probe includes an L511P probe, a Q513K probe, a Q513P probe, a D516V probe, a D516Y probe, an H526D probe, an H526L probe, an H526R probe, an H526Y probe, an S531L probe, an S531W probe, an L533P probe, or a combination thereof. The Q513K probe is not used together with the Q513P probe and L511P probe in a same PCR reaction, or the H526Y probe is not used together with the H526D probe, H526L probe, and H526R probe in another same PCR reaction.

In some embodiments, in the method for detecting drug resistance of Mycobacterium tuberculosis, the at least one detection probe includes: a first group of probes and a second group of probes. The first group of probes consists of the L511P probe, the Q513P probe, the H526D probe, the H526L probe, the H526R probe, and the S531W probe. The second group of probes consists of the Q513K probe, the D516V probe, the D516Y probe, the H526Y probe, the S531L probe, and the L533P probe. In the PCR reaction, at least one of the first group of probes is placed in the first PCR solution, and at least one of the second group of probes is placed in the second PCR solution.

In some embodiments, in the method for detecting drug resistance of Mycobacterium tuberculosis, the PNA blockers include: a first PNA blocker having the sequence of SEQ ID NO: 1; a second PNA blocker having the sequence of SEQ ID NO: 2; a third PNA blocker having the sequence of SEQ ID NO: 3; and a fourth PNA blocker having the sequence of SEQ ID NO: 4. In the PCR reaction, the first PCR solution includes the first PNA blocker, the third PNA blocker, and the fourth PNA blocker. The second PCR solution includes the first PNA blocker, the second PNA blocker, the third PNA blocker, and the fourth PNA blocker.

### BRIEF DESCRIPTION OF THE DRAWINGS

The present disclosure can be more fully understood by reading the following detailed description of the embodiment, with reference made to the accompanying drawings as follows.

FIG. 1A is a graph showing the amplification curves of the fluorescent quantitative PCR according to an example, used to evaluate the effect of PNA blockers on inhibiting non-specific amplification by closing the corresponding site.

FIG. 1B is a graph showing the amplification curves of the fluorescent quantitative PCR according to an example, used to evaluate the effect of the PNA blocker on inhibiting non-specific amplification by closing the corresponding sites.

FIG. 2 is a graph showing the amplification curve of the fluorescent quantitative PCR according to an example, used to evaluate the effect of the PNA blocker in inhibiting non-specific amplification by closing the corresponding site.

FIG. 3 is a graph showing the amplification curves of the fluorescent quantitative PCR according to an example, used to evaluate the inhibitory effects of different sequences of PNA blockers on non-specific amplification.

FIG. 4 is a graph showing the amplification curves of the fluorescent quantitative PCR according to an example, used to evaluate the inhibitory effects of adjusting the concentration of the PNA blocker on non-specific amplification.

FIG. 5 is a graph showing the amplification curve of fluorescent quantitative PCR according to an example, used to evaluate the optimization of the temperature program for quantitative PCR.

FIGS. 6A and 6B is a graph showing the amplification curves of the fluorescent quantitative PCR according to an example, used to examine the non-specific amplification results of the mutation detection probes under different combination conditions.

### DETAILED DESCRIPTION

The following will use drawings and detailed descriptions to clearly illustrate the spirit of the present disclosure. It should be understood that the present disclosure can have various modifications in different aspects, but these modifications do not depart from the scope of the present disclosure, and the descriptions and accompanying drawings are for illustrative purposes and are not intended to limit the present disclosure.

Embodiments of the present disclosure provide a plurality of PNA blockers for the wild-type template of the mutation sites in the 81 bp drug resistance determining region of the rpoB gene associated with rifampicin resistance in Mycobacterium tuberculosis. The combination of these PNA blockers can block the wild-type sequences of known mutation sites, such as codons 511, 513, 516, 526, 531, and 533, thereby largely or completely suppressing the non-specific amplification, which significantly enhances the specificity of the detection method.

Peptide nucleic acid (PNA) is a class of DNA analogs in which the sugar-phosphate backbone is replaced by a peptide backbone. Specifically, the neutral peptide chain is linked by amide bonds of 2-aminoethyl glycine, replacing the phosphodiester bonds found in DNA. The rest of the structure is similar to DNA structure. PNA can recognize and bind to DNA or RNA sequences through Watson-Crick base pairing, forming a stable double helix structure. Since PNA does not carry a negative charge, there is no electrostatic repulsion between PNA and DNA or RNA, which greatly enhances both the stability and specificity of the binding. Unlike the hybridization between DNA and DNA or between DNA and RNA, the hybridization of PNA with DNA or RNA is almost unaffected by the salt concentration of the hybridization system. The hybridization capability of PNA with DNA or RNA molecules is far superior to that of DNA/DNA or DNA/RNA, demonstrating high hybridization stability, excellent specific sequence recognition ability, resistance to hydrolysis by nucleases and proteases, and the ability to be co-transfected into cells when linked to ligands. These are advantages that other oligonucleotides do not possess.

The sequence of the oligonucleotide-like PNA has the following characteristics. (1) It cannot be recognized by DNA polymerase and has good self-stability. (2) The affinity and thermal stability of PNA/DNA are stronger than those of DNA/DNA, with high hybridization stability. (3) PNA/DNA is sensitive to base mismatches, and a single base mismatch can reduce the Tm value by 8° C. to 20° C. (average 15° C.). Therefore, in quantitative PCR methods for gene mutation detection, PNA sequences can be used as blockers.

In some embodiments, the plurality of PNA blockers are designed for mutation sites at codons 511, 513, 516, 526, 531, and 533 of rpoB gene. In some embodiments, two adjacent sites can be blocked by a same PNA blocker. In some embodiments, the sequence length of each of the PNA blockers is 14 to 18 bases. In some embodiments, the combination of blockers includes: a first PNA blocker targeting codons 511 and 513, a second PNA blocker targeting codon 516, a third PNA blocker targeting codon 526, and a fourth PNA blocker targeting codons 531 and 533.

In some embodiments, the sequence of the first PNA blocker is as shown in SEQ ID NO: 1, the sequence of the second PNA blocker is as shown in SEQ ID NO: 2, the sequence of the third PNA blocker is as shown in SEQ ID NO: 3, and the sequence of the fourth PNA blocker is as shown in SEQ ID NO: 4. Table 1 below shows the sequences of these PNA blockers and the corresponding codons, with the sequences of the corresponding codons indicated in bold and italic font.

TABLE 1

| SEQ | Corresponding | |
|---|---|---|
| ID NO | mutation site | 5′ to 3′ sequence |
| 1 | Codons 511 and 513 | CCAGCTGAGCCAAT |
| 2 | Codon 516 | TTCATGGACCAGAAC |
| 3 | codon 526 | CTTGTGGGTCAACC |
| 4 | Codons 531 and 533 | ACTGTCGGCGCTGG |

In some embodiments, the sequences of the PNA blockers may be derived sequences of SEQ ID NO: 1, SEQ ID NO: 2, SEQ ID NO: 3, or SEQ ID NO: 4. For example, one or two bases in length can be added to one or both ends of the sequences of SEQ ID NO: 1 to SEQ ID NO: 4, respectively. Therefore, the sequence of the first PNA blocker includes the sequence of SEQ ID NO: 1, the sequence of the second PNA blocker includes the sequence of SEQ ID NO: 2, the sequence of the third PNA blocker includes the sequence of SEQ ID NO: 3, and the sequence of the fourth PNA blocker includes the sequence of SEQ ID NO: 4. In other embodiments, the derived sequences may have one or two bases removed from one or both ends of each of the sequences of SEQ ID NO: 1 to SEQ ID NO: 4, and the position of the corresponding mutation site is not located at the very end of the blocker sequence. In yet other embodiments, 1 to 3 bases may be added to one end of the sequences of SEQ ID NO: 1 to SEQ ID NO: 4, while 1 or 2 bases may be removed from the other end, and the position of the corresponding mutation site is not located at the very end of the blocker sequence.

In some embodiments, in real-time fluorescent quantitative PCR, the combination of the PNA blockers is used to block the wild-type templates of the mutation sites in the drug resistance-determining region of rpoB gene in the sample. These PNA blockers are not modified with fluorescent groups. Additionally, in real-time fluorescent quantitative PCR, detection probes modified with fluorescent reporter groups are used simultaneously for the detection of mutation sites.

In some embodiments, a kit for detecting drug resistance of Mycobacterium tuberculosis is provided, including at least one set of primer pair, a combination of PNA blockers, and at least one mutation detection probe. The primer pair is used to amplify a segment of rpoB gene in PCR. The combination of PNA blockers is used to block the wild-type templates of known mutation sites in rpoB gene. The mutation detection probes are used to detect specific mutation sites. The sequences of the mutation detection probes can respectively match the specific mutation sites.

In some embodiments, the at least one set of primer pair in the kit may consist of one set of primer pair, two sets of primer pairs, or more sets of primer pairs. For example, when nested PCR is used for detection, two sets of primer pairs are employed: an outer primer pair and an inner primer pair. In some embodiments, the kit may also include a primer pair associated with an internal control plasmid.

In some embodiments, a plurality of mutation detection probes are used to simultaneously detect the mutation sites related to drug resistance in rpoB gene. The sequence of the blocker and the sequence of the detection probe are located on the same DNA strand of Mycobacterium tuberculosis genome, in order to block non-specific amplification in the presence of wild-type template sequences. Table 2 below shows the known drug resistance mutation sites in rpoB gene and the corresponding matching regions of the PNA blockers.

TABLE 2

| Codon of | | | |
|---|---|---|---|
| mutation | Mutation | Corresponding | Codons covered by |
| site | type | PNA blocker | the PNA blocker |
| codon 511 | L511P | First PNA Blocker | Codons 511 and 513 |
| codon 513 | Q513K | | |
| | Q513P | | |
| codon 516 | D516V | Second PNA Blocker | codon 516 |
| | D516Y | | |
| codon 526 | H526D | Third PNA Blocker | codon 526 |
| | H526L | | |
| | H526R | | |
| | H526Y | | |
| codon 531 | S531L | Fourth PNA blocker | Codons 531 and 533 |
| | S531W | | |
| codon 533 | L533P | | |

In some embodiments, corresponding mutation detection probes are designed for the 12 mutation types listed in Table 2 above, matching the mutated DNA template sequences. These mutation detection probes are a L511P probe, a Q513K probe, a Q513P probe, a D516V probe, a D516Y probe, a H526D probe, a H526L probe, a H526R probe, a H526Y probe, a S531L probe, a S531W probe, and a L533P probe. In some embodiments, the 5′ and 3′ ends of the mutation detection probes are modified with a fluorescent reporter group and a quencher group, respectively. The fluorescent reporter group may be, for example but not limited to, FAM, ROX, HEX, CY5, or the like.

In some embodiments, each of the mutation detection probes can be designed and prepared using methods known in the art. The sequence of the mutation detection probe may be, for example, 13 to 18 bases in length, with a Tm value of, for example, 65° C. to 80° C. In some embodiments, the probes can be prepared as TaqMan probes, TaqMan MGB probes, Molecular Beacon probes, or the like.

In some embodiments, the mutation detection probes used are shown in Table 3, where the sequence of the codons at the mutation sites is indicated in bold and italic font.

TABLE 3

| SEQ ID | | Mutation | |
|---|---|---|---|
| NO | Probe Name | type | 5′ to 3′ sequence |
| 14 | First probe | L511P | CAGCCGAGCCAAT |
| 15 | Second probe | Q513K | CTGAGCAAATTCATG |
| 16 | Third probe | Q513P | CTGAGCCCATTCAT |
| 17 | Fourth probe | D516V | AATTCATGGTCCAGAACA |
| 18 | Fifth probe | D516Y | TTCATGTACCAGAACAA |
| 19 | Sixth probe | H526D | CGCTTGTCGGTCAA |
| 20 | Seventh Probe | H526L | CTTGAGGGTCAACC |
| 21 | Eighth Probe | H526R | CTTGCGGGTCAAC |
| 22 | Ninth Probe | H526Y | CTTGTAGGTCAACCC |
| 23 | Tenth Probe | S531L | CCGACTGTTGGCG |
| 24 | Eleventh Probe | S531W | CGACTGTGGGCGC |
| 25 | Twelfth Probe | L533P | ACTGTCGGCGCCGG |

In some embodiments, multiple mutation detection probes can be used simultaneously for testing, such as the 12 types of mutation detection probes mentioned above, which can cover 98% of known cases of rifampicin resistance. The detection probes can be used individually or in combination in a PCR solution. In some embodiments, when multiple detection probes are used simultaneously, each of the detection probes can be added into an individual PCR container (e.g., a PCR tube or a well in a multi-well plate). In some embodiments, multiple mutation detection probes and the corresponding one or more PNA blockers can be added to a single PCR container. In some embodiments, when multiple mutation sites are detected in the same PCR container, the mutation detection probes can be respectively modified with different fluorescent reporter groups.

In some embodiments, the 12 mutation types of detection probes can be allocated to 2 to 4 containers (e.g., Eppendorf tubes), wherein each of the containers includes a respective combination of the detection probes, and then the reaction mixtures are added to the wells of a plate for automated processing. That is, some specific detection probes can be used together in the same PCR solution. In some embodiments, mixing certain mutation detection probes in the same reaction container may lead to an increase in non-specific amplification signals, so these mutation detection probes should be used in separate PCR solutions.

In some embodiments, the Q513K probe is not used in the PCR solution together with the Q513P probe and the L511P probe, meaning that the Q513K probe should be in a different PCR container from the Q513P probe and the L511P probe. For example, the Q513K probe is added into the first PCR tube, while the Q513P probe and the L511P probe are added into other PCR tubes different from the first PCR tube.

In some embodiments, the H526Y probe is not used in the PCR solution together with the H526D probe, H526L probe, and H526R probe, meaning that the H526Y probe should be in a different reaction container from the H526D probe, H526L probe, and H526R probe. For example, the H526Y probe is added into the second PCR tube, while the H526D probe, the H526L probe, and the H526R probe are added to other PCR tubes different from the second PCR tube.

In some embodiments, the 12 mutation types of the detection probes for detecting rifampicin resistance of Mycobacterium tuberculosis are divided into two PCR reaction mixtures; that is, the reagents are mixed separately in two tubes. The detection probes are divided into a first group of probes and a second group of probes. The first group of probes consists of the L511P probe, the Q513P probe, the H526D probe, the H526L probe, the H526R probe, and the S531W probe. The second group of probes consists of the Q513K probe, the D516V probe, the D516Y probe, the H526Y probe, the S531L probe, and the L533P probe. In the PCR detection, the probes belonging to the first group are mixed in the first PCR solution, and the probes belonging to the second group are mixed in the second PCR solution. Furthermore, depending on the combination of the probes added to the PCR solution, the corresponding PNA blockers are added into the PCR solution.

In some embodiments, quantitative PCR is performed using nested PCR to reduce non-specific amplification. The primer pairs in the kit for detecting drug resistance of Mycobacterium tuberculosis include an outer primer pair and an inner primer pair. Using two sets of primer pairs, two rounds of PCR amplification are performed. First, the outer primer pair is used for the initial amplification of the template DNA. Then, a small amount of the first reaction product is taken as the reaction template, and the inner primer pair is used for the second amplification. Using nested PCR approaches for continuous multi-round augmentation can increase the specificity and sensitivity of detection.

In some embodiments, the first round of PCR reaction is conducted for 8 to 15 cycles. The second round of PCR reaction is conducted for 35 to 50 cycles, wherein the annealing/extension stage of the second round of PCR reaction is divided into two steps, with the first step and the second step each performed at a temperature range of 60° C. to 75° C., and the temperature of the first step is higher than that of the second step. In some embodiments, the first step and the second step of the annealing/extension stage are each performed for 5 to 20 seconds, for example, 12 to 17 seconds. In other embodiments, the annealing/extension stage of the second round of PCR reaction is performed in one step, i.e., the temperature is maintained at a fixed temperature, and the time may be, for example, 5 to 30 seconds.

In some embodiments, the kit for detecting drug resistance of Mycobacterium tuberculosis further includes a plasmid, a set of primer pair, and a detection probe for internal control of the PCR detection.

In some embodiments, the kit for detecting drug resistance of Mycobacterium tuberculosis further includes reactants related to PCR reactions, such as related buffers, dNTPs, polymerase, etc.

The following provides examples for testing plasmids containing wild-type templates of resistance regions of rpoB, to specifically illustrate the embodiments of the present disclosure.

In the following examples, nested PCR was used, and the two sets of primer pairs for nested PCR are as shown in Table 4 below.

TABLE 4

| Sequence | | |
|---|---|---|
| Number | Prime Name | 5′ to 3′ sequence |
| SEQ ID NO: 7 | Forward external | AGGCGATCACACCGCAGACG |
| | primer | |
| SEQ ID NO: 8 | Reverse external | CCGACAGCGAGCCGATCAGA |
| | primer | |
| SEQ ID NO: 9 | Inner primer | CATCCGGCCGGTGGT |
| | forward | |
| SEQ ID NO: 10 | Internal primer | GGCACGCTCACGTGACAG |
| | reverse | |

In the following examples, the PCR solution includes a template plasmid as an internal control and associated primers and probes. The sequences of the primers and probes for detecting this template plasmid for internal control are as shown in Table 5 below.

TABLE 5

| Sequence | | |
|---|---|---|
| Number | Primer Name | 5′ to 3′ sequence |
| SEQ ID NO: 11 | Internal control | TGCGATCAGTAATTCAAAAC |
| | forward introduction | |
| SEQ ID NO: 12 | Internal control | GCTCAATCAACTCACTAATG |
| | reverse primer | |
| SEQ ID NO: 13 | Internal control | AACCACATACTTCCTGCCTTCATT |
| | probe | |

In some examples, a final fluorescence intensity of less than 50 units in PCR detection is considered as no non-specific amplification. In some examples, the Cq value (i.e., the number of amplification cycles required for the amplification product to reach the threshold) of the non-specific amplification signal is significantly delayed or the relative fluorescence intensity (RFU) is very low, and false-positive results can be excluded by algorithms.

### Example 1: Evaluation of the Effect of PNA Blockers on Inhibiting Non-Specific Amplification

In this example, the volume of the quantitative PCR solution was 25 microliters (μl), containing: master mix 1×, 200 nM each of the outer primer pairs for nested PCR, 300 nM of the inner forward primer, 200 nM of the inner reverse primer, 200 nM each of the internal control primer pairs, 100 nM of the internal control probe, 200 nM each of the mutation detection probes, 50 copies of the internal control template plasmid, 200 nM or 400 nM of the corresponding PNA blocker or MGB blocker, 10³ copies of the wild-type template, and molecular-grade water to make up to 25 μl. The sequences of the MGB blockers are the same as the sequences of the PNA blockers. The temperature setting of the quantitative PCR is shown in Table 6.

TABLE 6

| | | |
|---|---|---|
| temperature | time | Number of cycles |

| | | | |
|---|---|---|---|
| 95° C. | 2 | minutes | 1 |
| 95° C. | 15 | seconds | 10 |
| 65° C. | 30 | seconds | |
| 95° C. | 15 | seconds | 45 |
| 60° C. | 5 | seconds | |

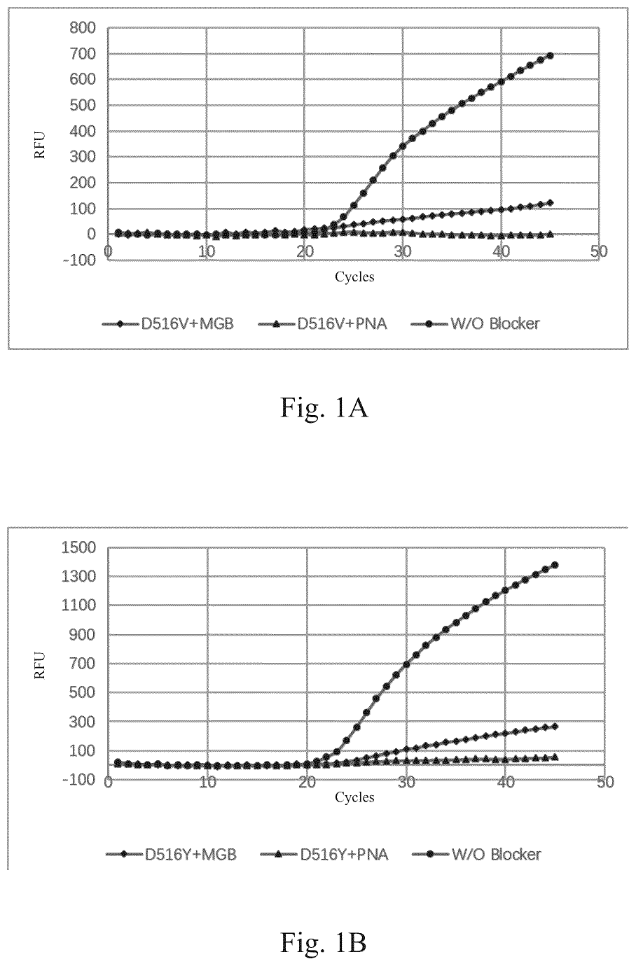

FIGS. 1A and 1B show the effect of the second PNA blocker of the embodiments of the present disclosure in suppressing non-specific amplification when present alone; i.e., only the blocker corresponding to codon 516 was placed in a PCR tube.

In the PCR tube related to FIG. 1A and the PCR tube related to FIG. 1B, the PNA blocker (indicated as PNA in the Figures) and MGB blocker (indicated as MGB in the Figures) used are the blockers corresponding to codon 516, i.e., the sequence is SEQ ID NO: 2, at a concentration of 200 nM. The mutation detection probe in the PCR tube related to FIG. 1A was the D516V probe, and the mutation detection probe in the PCR tube related to FIG. 1B was the D516Y probe.

Both FIGS. 1A and 1B show that in the PCR reaction without blockers (i.e., the curve of W/O Blocker), the final fluorescence intensity increases significantly, indicating the non-specific amplification of the D516V probe and the D516Y probe. As shown in FIG. 1A, the second PNA blocker corresponding to the mutation site of codon 516 can completely suppress the non-specific amplification caused by the D516V detection probe alone (see the curve of D516V+PNA). As shown in FIG. 1B, when the D516Y detection probe was present alone, the addition of the second PNA blocker still exhibited some degree of non-specific amplification (see the D516Y+PNA curve). Although this PNA blocker cannot completely suppress the non-specific amplification, the effect of reducing non-specific amplification was still evident. Additionally, both FIGS. 1A and 1B show that the PNA blockers exhibited a better suppression effect compared to the MGB blockers.

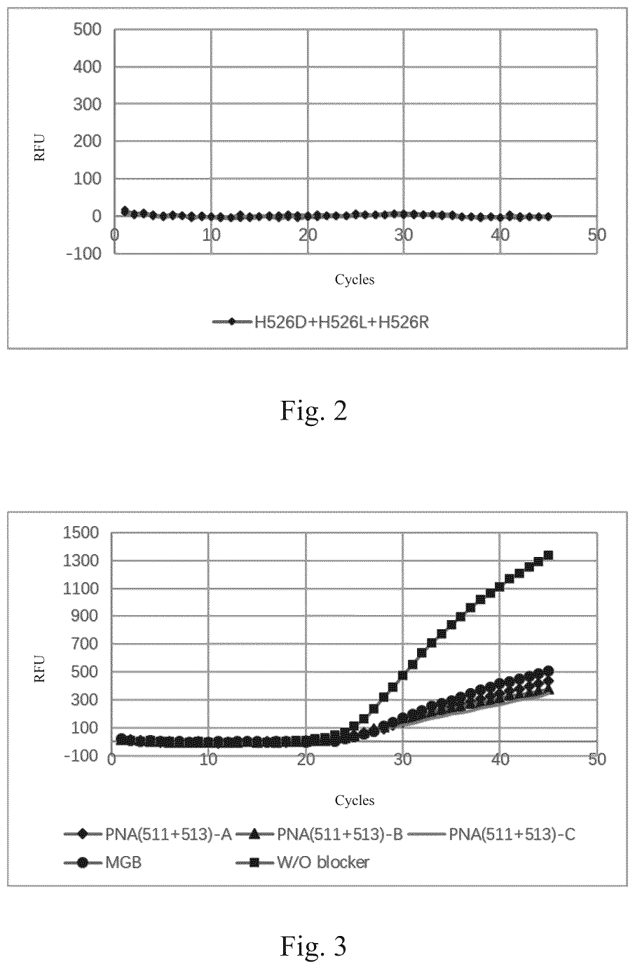

FIG. 2 shows the effect of the third PNA blocker corresponding to mutation site of codon 526 (sequence SEQ ID NO: 3) at a concentration of 400 nM in suppressing non-specific amplification when mixed with the H526D, H526L, and H526R mutation detection probes.

As seen in FIG. 2, the third PNA blocker, corresponding to the mutation site of codon 526, can completely suppress the non-specific amplification caused by the mixture of the three mutation detection probes.

### Example 2: Evaluation of the Non-Specific Amplification Inhibitory Effect of PNA Blockers Respectively Having Different Sequences

Taking the mutation sites at codons 511 and 513 as an example, three different sequences were designed for their corresponding wild-type templates, and the PNA blockers were synthesized. Under the same reagent system and temperature program, the non-specific amplification suppression effects of the three PNA blockers were examined. In FIG. 3, PNA blocker A has the sequence GCTGAGCCAATTCA, as shown in SEQ ID NO: 5; PNA blocker B has the sequence GCTGAGCCAATTCATG, as shown in SEQ ID NO: 6; and PNA blocker C has the sequence CCAGCTGAGCCAAT, which is the first PNA blocker mentioned earlier, as shown in SEQ ID NO: 1. In this example, the amount of wild-type template in each of PCR tubes was 10³ copies.

FIG. 3 shows the inhibitory effects of MGB blockers (identical to sequence SEQ ID NO: 1) and PNA blockers A, B, and C on non-specific amplification when mixed with detection probes for the mutation types L511P, Q513K, and Q513P. As seen in FIG. 3, PNA blockers A, B, and C exhibited a certain and significant inhibition of non-specific amplification compared to the case without any blockers, but the PNA blockers cannot completely suppress the non-specific amplification signal. Moreover, at the same concentration, the inhibitory effect of PNA blocker C was better than that of PNA blockers A and B.

### Example 3: Evaluation of the Effect of Adjusting the Concentration of PNA Blockers on the Inhibition of Non-Specific Amplification

In this example, the third PNA blocker corresponding to codon 526, with the sequence SEQ ID NO: 3, was used at concentrations of 200, 400, and 600 nM. When the third PNA blocker was mixed with the three detection probes for the mutation types L511P, Q513K, and Q513P, the inhibitory effect on non-specific amplification was observed.

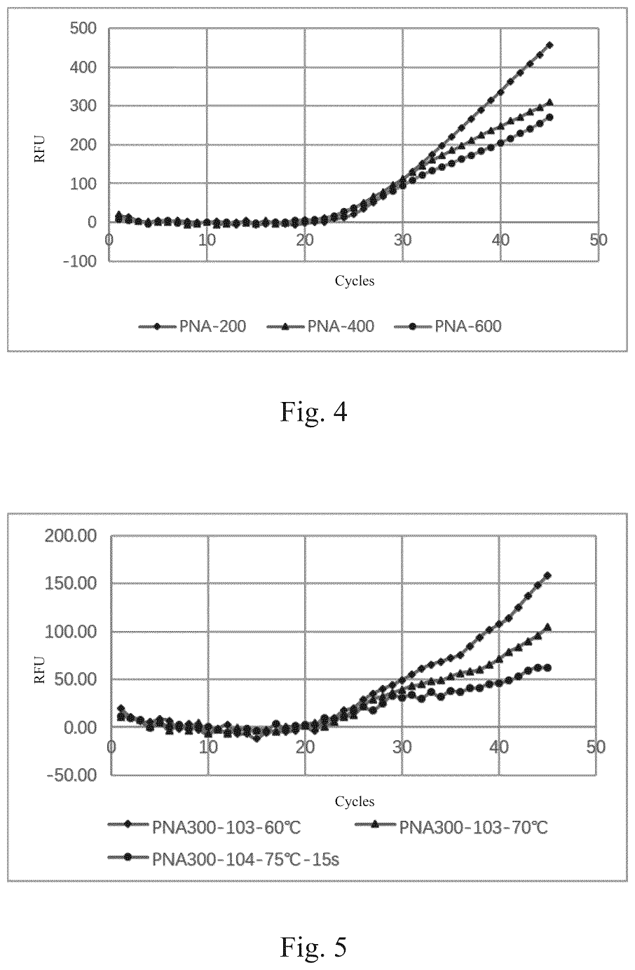

As seen in FIG. 4, as the concentration of the PNA blockers increased, the degree of suppression of non-specific amplification gradually was strengthened, resulting in a progressively lower endpoint fluorescence intensity; this indicates a reduced likelihood of interference with the amplification results for mutated templates. That is, when PCR detection is conducted, the degree of non-specific amplification of the detection probes on the wild-type template can be reduced by increasing the concentration of the PNA blockers.

### Example 4: Optimization of the Quantitative PCR Temperature Program

In this example, the annealing/extension stage of the second round of the nested PCR reaction was divided into two steps. The first step was tested at three different temperatures (60° C., 70° C., and 75° C.), and the second step was maintained at 60° C. The quantitative PCR temperature program for Example 4 is as shown in Table 7.

TABLE 7

| temperature | time | | Number of cycles |
|---|---|---|---|
| 95° C. | 2 | minutes | 1 |
| 95° C. | 15 | seconds | 10 |
| 65° C. | 30 | seconds | |
| 95° C. | 15 | seconds | 45 |
| 60° C./70° C./75° C. | 15 | seconds | |
| 60° C. | 15 | seconds | |

In this example, the third PNA blocker, corresponding to codon 526 and having the sequence SEQ ID NO: 3, was used, and the concentration was 300 nM. In a PCR tube, detection probes corresponding to four different mutation types H526D, H526L, H526R, and H526Y were mixed. In the second round of amplification, which primarily involved the internal primer pair, the temperature of the first step of the annealing and extension process was adjusted from the original 60° C. to 70° C. and 75° C., respectively, to examine the inhibitory effect of the PNA blockers on non-specific amplification at different temperatures of annealing/extension.

As shown in FIG. 5, as the temperature of the first step of the annealing/extension increased, the PNA blockers bound better to the wild-type template, resulting in a more pronounced inhibition of non-specific amplification.

### Example 5: Evaluation of Non-Specific Amplification of Mutant Detection Probes in Different Combinations

In this example, three detection probes corresponding to mutation types D516V, D516Y, and H526Y were mixed in one tube (Tube 1), along with the second PNA blocker corresponding to mutation site of codon 516 (SEQ ID NO: 2), and the third PNA blocker corresponding to mutation site of codon 526 (SEQ ID NO: 3); three detection probes for different mutation types H526D, H526L, and H526R at mutation site 526 were mixed in another tube (Tube 2), along with the corresponding third PNA blocker. This example is used to study whether the corresponding PNA blocker can completely inhibit non-specific amplification when the wild-type template is input at 10³ copies per reaction. Based on this, the non-specific amplification when the detection probe for the mutation type Q513K is mixed with the existing three probes in tube 1 and tube 2 was investigated. In other words, a mixture of four different mutation types of detection probes was present in each of the tubes, and the non-specific amplification of the different combinations of the above probes was evaluated.

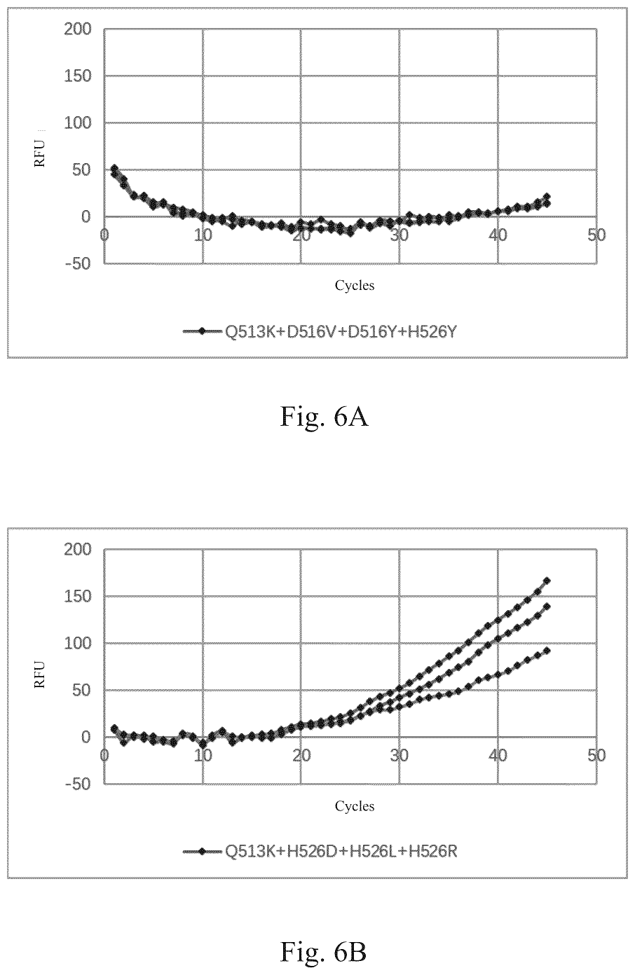

FIGS. 6A and 6B show the amplification curves of the wild-type template input of 10³ copies/reaction. FIG. 6A shows the amplification curves of the wild-type template mixed with the four detection probes corresponding to the mutation types Q513K, D516V, D516Y, and H526Y. FIG. 6B shows the amplification curves of the wild-type template mixed with four detection probes corresponding to the mutation types Q513K, H526D, H526L, and H526R.

As shown in FIG. 6A, when the Q513K detection probe was mixed with the three detection probes corresponding to the mutation types D516V, D516Y, and H526Y, the non-specific amplification signal essentially disappeared. As shown in FIG. 6B, when the Q513K detection probe was mixed with the three detection probes corresponding to the mutation types H526D, H526L, and H526R, there was still a slight non-specific amplification signal. This may be related to the interaction between different mutation detection probes.

The combination of the PNA blockers, the detection reagents, and the detection methods of the embodiments of the present disclosure can avoid or reduce the issue of non-specific amplification caused by wild-type templates at mutation sites during quantitative PCR detection. Moreover, the embodiments of the present disclosure can also be combined with sample pretreatment techniques for Mycobacterium tuberculosis and nucleic acid extraction reagents, allowing for direct application on commercial quantitative PCR machines. The embodiments can also be integrated into molecular point-of-care testing (POCT) detection cartridges, achieving simple, rapid, and accurate detection of rifampicin resistance of Mycobacterium tuberculosis, with broad application prospects.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention covers modifications and variations of this invention provided they fall within the scope of the following claims.

What is claimed is:

1. A combination of blockers for detecting drug resistance of Mycobacterium tuberculosis, comprising:
a first peptide nucleic acid (PNA) blocker covering codon 511 and codon 513 of rpoB gene;
a second PNA blocker covering codon 516 of the rpoB gene;
a third PNA blocker covering codon 526 of the rpoB gene; and
a fourth PNA blocker covering codon 531 and codon 533 of the rpoB gene;
wherein sequences of the first, second, third, and fourth PNA blockers match wild-type sequences of corresponding regions of the rpoB gene.

2. The combination of blockers for detecting drug resistance of Mycobacterium tuberculosis of claim 1, wherein the first PNA blocker comprises a sequence of SEQ ID NO: 1 or a derivative thereof, the second PNA blocker comprises a sequence of SEQ ID NO: 2 or a derivative thereof, the third PNA blocker comprises a sequence of SEQ ID NO: 3 or a derivative thereof, and the fourth PNA blocker comprises a sequence of SEQ ID NO: 4 or a derivative thereof.

3. The combination of blockers for detecting drug resistance of Mycobacterium tuberculosis of claim 1, wherein each of the first, second, third, and fourth PNA blockers has 14 to 18 bases.

4. The combination of blockers for detecting drug resistance of Mycobacterium tuberculosis of claim 1, wherein a sequence of the first PNA blocker is SEQ ID NO: 1, a sequence of the second PNA blocker is SEQ ID NO: 2, a sequence of the third PNA blocker is SEQ ID NO: 3, and a sequence of the fourth PNA blocker is SEQ ID NO: 4.

5. The combination of blockers for detecting drug resistance of Mycobacterium tuberculosis of claim 1, wherein the first, second, third, and fourth PNA blockers do not contain fluorescent modification groups.

6. A kit for detecting drug resistance of Mycobacterium tuberculosis, comprising:
at least one set of primer pair, for amplifying a segment of rpoB gene;
a combination of blockers, comprising a plurality of PNA blockers covering codon 511, codon 513, codon 516, codon 526, codon 531, and codon 533 in a drug resistance-determining region of the rpoB gene, wherein sequences of the plurality of PNA blockers respectively match wild-type sequences of corresponding regions; and
at least one mutation detection probe for detecting a mutation in at least one of the codon 511, the codon 513, the codon 516, the codon 526, the codon 531, and the codon 533 in the drug resistance-determining region of the rpoB gene.

7. The kit for detecting drug resistance of Mycobacterium tuberculosis of claim 6, wherein the at least one set of primer pair comprises two sets of primer pairs for nested PCR.

8. The kit for detecting drug resistance of Mycobacterium tuberculosis of claim 6, wherein the plurality of PNA blockers comprise:
a first PNA blocker covering the codon 511 and the codon 513 of the rpoB gene;
a second PNA blocker covering the codon 516 of the rpoB gene;
a third PNA blocker covering the codon 526 of the rpoB gene; and
a fourth PNA blocker covering the codon 531 and the codon 533 of the rpoB gene.

9. The kit for detecting drug resistance of Mycobacterium tuberculosis of claim 8, wherein the first PNA blocker comprises a sequence of SEQ ID NO: 1 or a derivative thereof, the second PNA blocker comprises a sequence of SEQ ID NO: 2 or a derivative thereof, the third PNA blocker comprises a sequence of SEQ ID NO: 3 or a derivative thereof, and the fourth PNA blocker comprises a sequence of SEQ ID NO: 4 or a derivative thereof.

10. The kit for detecting drug resistance of Mycobacterium tuberculosis of claim 8, wherein a sequence of the first PNA blocker is SEQ ID NO: 1, a sequence of the second PNA blocker is SEQ ID NO: 2, a sequence of the third PNA blocker is SEQ ID NO: 3, and a sequence of the fourth PNA blocker is SEQ ID NO: 4.

11. The kit for detecting drug resistance of Mycobacterium tuberculosis of claim 6, wherein each of the at least one mutation detection probe is used to detect a mutation site in the rpoB gene, and the mutation site is one of L511P, Q513K, Q513P, D516V, D516Y, H526D, H526L, H526R, H526Y, S531L, S531W, and L533P.

12. The kit for detecting drug resistance of Mycobacterium tuberculosis of claim 6, wherein the plurality of PNA blockers do not contain fluorescent groups, and a 5′ end and a 3′ end of the at least one mutation detection probe are respectively modified with a fluorescent reporter group and a quencher group.

13. A method for detecting drug resistance of Mycobacterium tuberculosis, comprising:
adding template DNA, at least one set of primer pair, a combination of blockers, and at least one mutation detection probe to a reaction system to perform a PCR reaction, wherein the combination of blockers comprises a plurality of PNA blockers covering codon 511, codon 513, codon 516, codon 526, codon 531, and codon 533 in a drug resistance-determining region of rpoB gene, and sequences of the plurality of PNA blockers respectively match wild-type sequences of corresponding regions, and the at least one mutation detection probe is used to detect a mutation site in at least one of the codon 511, the codon 513, the codon 516, the codon 526, the codon 531, and the codon 533 in the drug resistance-determining region of the rpoB gene; and
determining whether the template DNA has a rifampicin resistance related mutation based on reaction signals from the PCR reaction.

14. The method for detecting drug resistance of Mycobacterium tuberculosis of claim 13, wherein the PCR reaction is a nested PCR, comprising:
a first round of PCR reaction conducted for 8 to 15 cycles; and
a second round of PCR reaction conducted for 35 to 50 cycles, wherein an annealing/extension stage of the second round of PCR reaction is divided into a first step and a second step, and a temperature of the first step is higher than a temperature of the second step.

15. The method for detecting drug resistance of Mycobacterium tuberculosis of claim 13, wherein the PNA blockers comprise:
a first PNA blocker covering the codon 511 and the codon 513 of the rpoB gene;
a second PNA blocker covering the codon 516 of the rpoB gene;
a third PNA blocker covering the codon 526 of the rpoB gene; and
a fourth PNA blocker covering the codon 531 and the codon 533 of the rpoB gene.

16. The method for detecting drug resistance of Mycobacterium tuberculosis of claim 15, wherein the first PNA blocker comprises a sequence of SEQ ID NO: 1 or a derivative thereof, the second PNA blocker comprises a sequence of SEQ ID NO: 2 or a derivative thereof, the third PNA blocker comprises a sequence of SEQ ID NO: 3 or a derivative thereof, and the fourth PNA blocker comprises a sequence of SEQ ID NO: 4 or a derivative thereof.

17. The method for detecting drug resistance of Mycobacterium tuberculosis of claim 13, wherein each of the at least one mutation detection probe is used to detect the mutation site in the rpoB gene, and the mutation site is one of L511P, Q513K, Q513P, D516V, D516Y, H526D, H526L, H526R, H526Y, S531L, S531W, and L533P.

18. The method for detecting drug resistance of Mycobacterium tuberculosis of claim 17, wherein:
the at least one mutation detection probe comprises an L511P probe, a Q513K probe, a Q513P probe, a D516V probe, a D516Y probe, an H526D probe, an H526L probe, an H526R probe, an H526Y probe, an S531L probe, an S531W probe, an L533P probe, or a combination thereof; and
wherein the Q513K probe is not used together with the Q513P probe and the L511P probe in a same container of the PCR reaction; or
the H526Y probe is not used together with the H526D probe, the H526L probe, and the H526R probe in another same container in the PCR reaction.

19. The method for detecting drug resistance of Mycobacterium tuberculosis of claim 18, wherein the at least one mutation detection probe comprises:
a first group of probes consisting of the L511P probe, the Q513P probe, the H526D probe, the H526L probe, the H526R probe, and the S531W probe; and
a second group of probes consisting of the Q513K probe, the D516V probe, the D516Y probe, the H526Y probe, the S531L probe, and the L533P probe;
wherein in the PCR reaction, at least one of the first group of probes is placed in a first PCR solution, and at least one of the second group of probes is placed in a second PCR solution.

20. The method for detecting drug resistance of Mycobacterium tuberculosis of claim 19, wherein the plurality of PNA blockers comprise:
a first PNA blocker having a sequence of SEQ ID NO: 1;
a second PNA blocker having a sequence of SEQ ID NO: 2;
a third PNA blocker having a sequence of SEQ ID NO: 3; and
a fourth PNA blocker having a sequence of SEQ ID NO: 4;
wherein in the PCR reaction, the first PCR solution includes the first PNA blocker, the third PNA blocker, and the fourth PNA blocker, and the second PCR solution includes the first PNA blocker, the second PNA blocker, the third PNA blocker, and the fourth PNA blocker.


SEQUENCE LISTING (25 sequences)

SEQ ID NO: 1 - DNA, 14 bp, synthetic construct
  source 1..14: mol_type = other DNA
ccagctgagccaat

SEQ ID NO: 2 - DNA, 15 bp, synthetic construct
  source 1..15: mol_type = other DNA
ttcatggaccagaac

SEQ ID NO: 3 - DNA, 14 bp, synthetic construct
  source 1..14: mol_type = other DNA
cttgtgggtcaacc

SEQ ID NO: 4 - DNA, 14 bp, synthetic construct
  source 1..14: mol_type = other DNA
actgtcggcgctgg

SEQ ID NO: 5 - DNA, 14 bp, synthetic construct
  source 1..14: mol_type = other DNA
gctgagccaattca

SEQ ID NO: 6 - DNA, 16 bp, synthetic construct
  source 1..16: mol_type = other DNA
gctgagccaattcatg

SEQ ID NO: 7 - DNA, 20 bp, synthetic construct
  source 1..20: mol_type = other DNA
aggcgatcacaccgcagacg

SEQ ID NO: 8 - DNA, 20 bp, synthetic construct
  source 1..20: mol_type = other DNA
ccgacagcgagccgatcaga

SEQ ID NO: 9 - DNA, 15 bp, synthetic construct
  source 1..15: mol_type = other DNA
catccggccggtggt

SEQ ID NO: 10 - DNA, 18 bp, synthetic construct
  source 1..18: mol_type = other DNA
ggcacgctcacgtgacag

SEQ ID NO: 11 - DNA, 20 bp, synthetic construct
  source 1..20: mol_type = other DNA
tgcgatcagtaattcaaaac

SEQ ID NO: 12 - DNA, 20 bp, synthetic construct
  source 1..20: mol_type = other DNA
gctcaatcaactcactaatg

SEQ ID NO: 13 - DNA, 24 bp, synthetic construct
  source 1..24: mol_type = other DNA
aaccacatacttcctgccttcatt

SEQ ID NO: 14 - DNA, 13 bp, synthetic construct
  source 1..13: mol_type = other DNA
cagccgagccaat

SEQ ID NO: 15 - DNA, 15 bp, synthetic construct
  source 1..15: mol_type = other DNA
ctgagcaaattcatg

SEQ ID NO: 16 - DNA, 14 bp, synthetic construct
  source 1..14: mol_type = other DNA
ctgagcccattcat

SEQ ID NO: 17 - DNA, 18 bp, synthetic construct
  source 1..18: mol_type = other DNA
aattcatggtccagaaca

SEQ ID NO: 18 - DNA, 17 bp, synthetic construct
  source 1..17: mol_type = other DNA
ttcatgtaccagaacaa

SEQ ID NO: 19 - DNA, 14 bp, synthetic construct
  source 1..14: mol_type = other DNA
cgcttgtcggtcaa

SEQ ID NO: 20 - DNA, 14 bp, synthetic construct
  source 1..14: mol_type = other DNA
cttgagggtcaacc

SEQ ID NO: 21 - DNA, 13 bp, synthetic construct
  source 1..13: mol_type = other DNA
cttgcgggtcaac

SEQ ID NO: 22 - DNA, 15 bp, synthetic construct
  source 1..15: mol_type = other DNA
cttgtaggtcaaccc

SEQ ID NO: 23 - DNA, 13 bp, synthetic construct
  source 1..13: mol_type = other DNA
ccgactgttggcg

SEQ ID NO: 24 - DNA, 13 bp, synthetic construct
  source 1..13: mol_type = other DNA
cgactgtgggcgc

SEQ ID NO: 25 - DNA, 14 bp, synthetic construct
  source 1..14: mol_type = other DNA
actgtcggcgccgg